(19) United States
(12) Patent Application Publication
(10) Pub. No.: US 2010/0137230 A1
(43) Pub. Date: Jun. 3, 2010

(54) NEW PYRIDIN-2-ONE COMPOUNDS

(76) Inventor: Lutz Weber, Germering (DE)

Correspondence Address: DANN, DORFMAN, HERRELL & SKILLMAN, 1601 MARKET STREET, SUITE 2400, PHILADELPHIA, PA, 19103-2307, US

(21) Appl. No.: 12/696,365

(22) Filed: Jan. 29, 2010

Related U.S. Application Data
(63) Continuation of application No. 12/017,146, filed on Jan. 21, 2008, now abandoned.
(60) Provisional application No. 60/885,776, filed on Jan. 19, 2007.

(51) Int. Cl. A61K 31/4375; C07H 7/06; C07D 471/04; A61K 31/706; A61K 31/519; A61P 31/04

(52) U.S. Cl. 514/23; 536/53; 546/138; 544/282; 514/306; 514/259.41

(57) ABSTRACT

The present invention relates to compounds of the formula (I) that are useful antimicrobial agents and effective against a variety of multi-drug resistant bacteria:

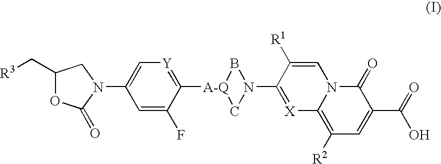

### CROSS-REFERENCE TO RELATED APPLICATIONS

The present application claims the benefit of U.S. Provisional Patent Application No. 60/885,776, filed Jan. 19, 2007, the entire disclosure of which is incorporated by reference herein.

### FIELD OF THE INVENTION

The present invention relates to novel compounds having antibacterial activity, pharmaceutical compositions containing such compounds, methods of treatment using such compounds and methods for synthesis of such compounds. These compounds combine at least a twofold mechanism of action by acting for example on bacterial topoisomerases and bacterial protein synthesis and are effective against a variety of human and veterinary pathogens including Gram positive aerobic bacteria such as multiple-resistant Staphylococci, Streptococci, Bacillus anthracis and Enterococci as well as Gram negative bacteria such as Escherichia coli, Moraxella catarrhalis and Haemophilus influenzae and anaerobic organisms such as Bacteroides spp. and Clostridia spp. species as well as acid resistant organisms such as Mycobacterium tuberculosis, Mycobacterium avium spp. Examples for resistant strains are Staphylococci resistant against Methicillin (MRSA), Staphylococcus epidermis resistant against Methicillin (MRSE), Staphylococcus pneumoniae resistant against Penicillin (PRSP) and Enterococci resistant against Vancomycin (VRE).

The present invention describes new compounds in which the antibacterial pharmacophores of pyridin-2-one, in particular the quinolizin-4-one and pyrido[1,2-a]pyrimidin-6-one moiety, and oxazolidinone are linked together through a chemically stable linker, forming a hybrid compound that contains both pharmacophores. Hybrid antibiotics which contain either the known quinolin-4-one or naphthyridin-4-one and the oxazolidinone moiety (Antimicrobial Agents and Chemotherapy, 1999, p. 1469-1474; Antimicrobial Agents: Antibacterials and Antifungals, Andre Bryskier, Ed. ASM Press, Washington, USA, 2005) have already been described (WO02059116, WO03002560, WO03031441, WO03031443, WO03032962, WO04096221, WO05023801; Bioorg. Med. Chem. Lett. 2003, 11(10), p. 2313-2319; Bioorg. Med. Chem. Lett. 2003, 13(23), p. 4213-4216). One drawback of the compounds known in the state of the art is their poor water solubility, which makes the development of a pharmaceutical formulation difficult. Therefore, methods were developed to overcome the problem of insufficient water solubility by introducing very polar water-soluble groups like sulfate or phosphate (WO05058888; C. Gray et al. “Efficacy Studies of MCB-3837, a Dual-action Antibiotic, in Experimental Infections in Mice”, ICAAC Meeting, Washington D.C., USA, Dec. 16-19, 2005). However, these groups are cleaved very fast in serum releasing sulfate or phosphate into the blood stream, which is physiologically of disadvantage. Additionally, a fast precipitation of the administered drug substance can occur after the cleavage of the solubilising group where the injection occurred being a cause for potential further adverse side effects.

A further drawback of hybrid antibiotics known in the state of the art and composed of quinolin-4-ones or naphthyridin-4-one and oxazolidinones is their reduced ability to inhibit Gram-negative bacteria when compared with related quinolin-4-one or naphthyridin-4-one antibiotics that are not hybrid antibiotics.

Therefore, a solution is provided in such a way that the quinolin-4-one or naphthyridin-4-one moiety in the hybrid antibiotics known in the state of the art is replaced by a quinolizin-4-one or pyrido[1,2-a]pyrimidin-6-one moiety, respectively, both sharing the pyridin-2-one substructure and resulting in oxazolidinone-pyridin-2-one hybrid antibiotics. According to the present invention, these compounds have a high in vitro activity against a variety of bacterial strains as well as a better aqueous solubility.

Further, another solution is provided in such a way that by chemical attachment of a monosaccharide, disaccharide, oligosaccharide or polysaccharide moiety onto the hybrid antibiotics according to the present invention, their hydrophilicity and the water solubility of new compounds of formula (I) is significantly enhanced and therefore, not only the physico-chemical properties are improved but also the pharmacological properties such as the halftime in the blood and adverse side effects are reduced.

### SUMMARY OF THE INVENTION

The present invention provides new compounds of formula (I), that are useful antimicrobial agents and effective against a variety of multi-drug resistant bacteria,

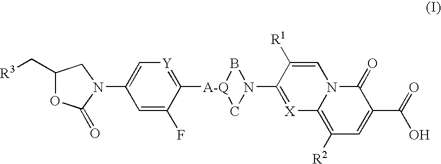

wherein
R¹ is H or F; and
R² is an alkyl group, an alkenyl group, an alkynyl group, a heteroalkyl group, a cycloalkyl group, a heterocyclo-alkyl group, an aryl group, a heteroaryl group, an alkyl-aryl group or a heteroarylalkyl group; all of which may be substituted with one, two or more halogen atoms like F or Cl or hydroxy or amino groups; and
R³ is an azido, or a C₁₋₆-heteroalkyl group, a heteroarylalkyl group, a heteroarylcycloalkyl group or a heteroalkylheteroaryl group; and
A is a single bond, O, S, S(═O), SO₂ or an alkylene group, an alkenylene group, an alkynylene group, a heteroalkylene group, a cycloalkylene group, a heterocycloalkylene group, an arylene group or a heteroarylene group all of which groups may be substituted; and
B and C are independent from each other alkylene, alkenylene, alkynylene or heteroalkylene, whereby Q-B—N—C are forming together a heterocycloalkyl group or a bicyclic heterocycloalkyl group, all of these groups may be substituted with one or more R⁴ groups; and
Q is CR⁴ or N; and
X is CR⁵ or N; and
Y is CH, CF or N; and
R⁴ is H, OR⁶, a group of formula —OPO₃R⁶₂ or —OSO₃R⁶ or an alkyl group or a heteroalkyl group carrying one or more OR⁶, —OPO₃R⁶₂ or —OSO₃R⁶ group(s), wherein the groups R⁶ independently of each other are H, an ether or an ester of a natural or unnatural, substituted or unsubstituted monosaccharide, a natural or unnatural, substituted or unsubstituted disaccharide, a natural or unnatural, substituted or unsubstituted oligosaccharide or a natural or unnatural, substituted or unsubstituted polysaccharide.
R⁵ is H, CH₃, OCH₃, F, Cl, OH, NH₂, —CN, an alkyl group or a heteroalkyl group, and
R² and R⁵ can be linked via an alkylene, an alkenylene or a heteroalkylene group or be a part of a cycloalkylene or heterocycloalkylene group, in case R² is not H and R⁵ is not H, CH₃, OCH₃, F, Cl, OH, NH₂, —CN;
or a pharmacologically acceptable salt, solvate, hydrate or formulation thereof.

It should be appreciated that certain compounds of formula (I) may have tautomeric forms from which only one might be specifically mentioned or depicted in the following description, additionally different geometrical isomers which are usually denoted as cis/trans isomers or more generally as (E) and (Z) isomers, or different optical isomers as a result of one or more chiral carbon atoms (which are usually nomenclatured under the Cahn-Ingold-Prelog or R/S system) might occur. Furthermore, the mono-, di-, oligo- or polysaccharides may occur in one or more different anomeric forms (e.g. alpha-D-glucuronic acid or beta-D-glucuronic acid). Further, some compounds may display polymorphism. All these tautomeric forms, geometrical or optical isomers (as well as racemates, anomers and diastereomers) and polymorphous forms are included in this invention.

The term alkyl refers to a saturated straight or branched chain alkyl group, containing from one to ten, preferably from one to six carbon atoms, for example methyl, ethyl, propyl, isopropyl, butyl, iso-butyl, sec-butyl, tert-butyl, n-pentyl, iso-pentyl, n-hexyl, 2,2-dimethylbutyl, n-heptyl, n-octyl groups. Any alkyl group as defined herein may be substituted with one, two or more substituents, for example F, Cl, Br, I, CN, NH₂, OH, SH or NO₂.

The terms alkenyl and alkynyl refer to a unsaturated straight or branched chain alkyl group (having one, two or more double and/or triple bonds, an alkenyl preferably having one or two double bonds and an alkynyl preferably having one or two triple bonds), containing two to ten, preferably two to six carbon atoms for example: ethenyl (vinyl), propenyl (allyl), iso-propenyl, n-pentenyl, butenyl, isoprenyl or hexa-2-enyl; ethynyl, propynyl or butynyl groups. Any alkenyl or alkynyl group as defined herein may be substituted with one, two or more substituents, for example F, Cl, Br, I, CN, NH₂, OH, SH or NO₂.

The term heteroalkyl refers to an alkyl, alkenyl or alkynyl group as defined herein where one or more carbon atoms are replaced by an oxygen, nitrogen, phosphorus or sulphur atom for example an alkoxy group such as methoxy, ethoxy, propoxy, iso-propoxy, butoxy or tert.-butoxy, an alkoxyalkyl group such as methoxymethyl, ethoxymethyl, 1-methoxyethyl, 1-ethoxyethyl, 2-methoxyethyl or 2-ethoxyethyl, an alkylamino group such as methyl amino, ethyl amino, propyl amino, isopropyl amino, dimethyl amino or diethyl amino, an alkyl thio group such as methylthio, ethylthio or isopropylthio or a cyano group. It may also refer to one of the above groups containing a keto group. The term heteroalkyl furthermore refers to a group derived from a carboxylic acid or carboxylic acid amide such as acetyl, propionyl, acetyloxy, propionyloxy, acetyl amino or propionyl amino, a carboxy alkyl group such as carboxymethyl, carboxy ethyl or carboxy propyl, a carboxy alkyl ester, an alkylthiocarboxyamino group, an alkoxyimino group, an alkylaminothiocarboxyamino group or an alkoxycarbonylamino group. Any heteroalkyl group as defined herein may be substituted with one, two or more substituents, for example F, Cl, Br, I, CN, NH₂, OH, SH or NO₂.

The term cycloalkyl refers to a saturated or partially unsaturated (having one, two or more double and/or triple bonds), cyclic group with one, two or more rings, having three to 14 carbon ring-atoms, preferably from five or six to ten carbon ring-atoms, for example cyclopropyl, cyclobutyl, cyclopentyl, cyclohexyl, tetralin, cyclopentenyl or cyclohex-2-enyl groups. Any cycloalkyl group as defined herein may be substituted with one, two or more substituents, for example F, Cl, Br, I, OH, NH₂, SH, N₃, NO₂, alkyl groups such as methyl or ethyl, heteroalkyl groups such as methoxy, methylamino, dimethylamino or cyanide.

The term heterocycloalkyl refers to a cycloalkyl group as defined herein where one, two or more carbon ring-atoms are replaced by one, two or more oxygen, nitrogen, phosphorus or sulphur atoms or S(0)₁₋₂ groups for example piperidino, morpholino or piperazino groups.

The term aryl refers to an aromatic cyclic group with one, two or more rings, having five to 14 carbon ring-atoms preferably from five or six to ten carbon ring-atoms, for example phenyl or naphthyl groups. Any aryl group as defined herein may be substituted with one, two or more substituents, for example F, Cl, Br, I, OH, NH₂, SH, N₃, NO₂, alkyl groups such as methyl or ethyl, heteroalkyl groups such as methoxy, methylamino, dimethylamino or cyanide.

The term heteroaryl refers to an aryl group as defined herein where one, two or more ring-carbon atoms are replaced by an oxygen, nitrogen, boron, phosphorus or sulphur atom, for example pyridyl, imidazolyl, pyrazolyl, quinolinyl, isoquinolinyl, pyrrolyl, oxazolyl, isoxazolyl, thiazolyl, isothiazolyl, 1,2,3-triazolyl, 1,2,4-triazolyl, oxadiazolyl, thiadiazolyl, indolyl, indazolyl, tetrazolyl, pyrazinyl, pyrimidinyl and pyridazinyl groups.

The term aralkyl (or arylalkyl or alkylaryl) refers to groups that comprise both aryl and as well as alkyl and/or cycloalkyl groups.

The term heteroarylalkyl (or heteroalkylaryl or heteroalkylheteroaryl etc.) refers to an aralkyl group as defined herein where one, two, three or more carbon atoms are replaced by one, two, three or more oxygen, nitrogen, phosphorus or sulfur atoms or S(O)₁₋₂ groups.

Any alkyl, alkenyl, alkynyl, heteroalkyl, cycloalkyl, heterocycloalkyl, aryl, heteroaryl, aralkyl or heteroarylalkyl groups as defined herein may be substituted with one or more halogen atoms, CN, NH₂, SH, NO₂ or OH groups or unsubstituted alkyl, heteroalkyl, aryl, aralkyl, aralkyloxy, heteroaryl, cycloalkyl or heterocycloalkyl groups as defined herein.

The terms “optionally substituted” or “substituted” refer to groups wherein one or more hydrogen atoms may be replaced by a halogen atom, a NH₂, SH, NO₂ or OH group or by an unsubstituted alkyl, heteroalkyl, aryl, aralkyl, aralkyloxy, heteroaryl, cycloalkyl or heterocycloalkyl group as defined herein.

The term natural or unnatural, substituted or unsubstituted monosaccharide refers to aldoses and ketoses of trioses, tetroses, pentoses, hexoses and heptoses. Examples are glucose, glucosamine, mannose, allose, galactose, fructose, ribose, arabinose, xylose, streptose, apiose etc. and their respective oxidized or substituted derivatives such as glucuronic acid, mannopyranuronic acid, gluco-pyranosiduronic acid, tartaric acid, xylaric acid, or galactaric acid (definitions of monosaccharides are also found in standard chemistry textbooks, for example “Monosaccharides: Their Chemistry and Their Roles in Natural Products” Peter M. Collins and Robert J. Ferrier, John Wiley & Sons, 1998, or “The organic chemistry of sugars” ed. by Daniel E. Ley and Peter Fugedi, CRC/Taylor & Francis, 2006).

The term natural or unnatural, substituted or unsubstituted di- or oligosaccharides refers to saccharides that are formed by 2-8 monosaccharides. The most common are disaccharides that are formed by the same two monosaccharides or different ones (definitions are also found in standard chemistry text books). Examples are trehalose, maltose, saccharose, lactose etc. and their respective oxidized or substituted derivatives and pegylated oligosaccharides.

The term natural or unnatural, substituted or unsubstituted polysaccharide refers to molecules, which are formed by a multitude of the same monosaccharides (homopolysaccharides), or of two or more different monosaccharides (heteropoly-saccharides) and their respective oxidized or substituted derivatives. The structure of the polysaccharides can be linear, branched or cyclic. Examples are dextrine, cyclo-dextrines, glycogen, starch, cellulose, modified polysaccharides such as hydroxy ethyl starch (HES), or pegylated polysaccharides.

### DESCRIPTION OF PREFERRED EMBODIMENTS

Preferred and/or advantageous embodiments of the invention are subject matter of the subclaims.

Preferred are compounds of formula (I), wherein R¹ is F.

Moreover preferred are compounds of formula (I), wherein R² is ethyl, 2-propyl, cyclopropyl, cyclobutyl, cyclopentyl or cyclohexyl, a phenyl or a pyridyl group. All these groups may be substituted with one, two or more fluorine atoms or amino groups.

Especially preferred are compounds of formula (I), wherein R² is a cyclopropyl group.

Further preferred are compounds of formula (I), wherein R² and R⁵ together form a bridge of the formula —O—CH₂—N(Me)-, —O—CH₂CH₂— or —O—CH₂CH(Me)-. Herein, the preferred stereochemistry at the chiral center is the one giving the S configuration in the final compound.

Moreover preferred are compounds of formula (I), wherein R⁴ is H, OR⁶, or a group of formula OPO₃R⁶₂ or OSO₃R⁶ or a alkyl group or a heteroalkyl group carrying one or more OR⁶, —OPO₃R⁶₂ or OSO₃R⁶ group(s), wherein the groups R⁶ independently of each other are H, an ether or an ester of a natural or unnatural, substituted or unsubstituted monosaccharide, a natural or unnatural, substituted or unsubstituted disaccharide, a natural or unnatural, substituted or unsubstituted oligo-saccharide or a natural or unnatural, substituted or un-substituted polysaccharide.

Further preferred are compounds of formula (I), wherein R³ is a group of the formula —NHCOCH═CH-aryl, heteroaryl such as unsubstituted 1,2,3-triazol or 1,2,3-triazol substituted by F, Cl or Me, or -oxa-3-oxazole, —NHSO₂Me, —NHSOMe, —NHCOOMe, —NHCOMe, —NHCS₂Me, —NHCSMe, —NHCSNH₂, or —NHCSOMe.

Especially preferred are compounds of formula (I), wherein R³ is —NHCOMe, —NHCSMe, —NHCOCHF₂, or —NHCOCHCl₂.

Further preferred are compounds of formula (I), wherein X is CH, CMe or N.

Moreover preferred are compounds of formula (I), wherein Y is CH, CF, N or a methoxy group, which may be substituted by one, two or three fluorine atoms.

Further preferred are compounds of formula (I), wherein A is O, S, S(═O), SO₂, C₂₋₆ alkylene, C₂₋₆ alkenylene, C₂₋₆ alkynylene, C₁₋₆ heteroalkylene, cyclopropylene, epoxide, aziridine, thioepoxide, lactame or lactone, all of which groups may be substituted.

Moreover preferred are compounds of formula (I), wherein A is a group of formula —O-D-, wherein D is a C₁₋₄ alkylene group, a C₂₋₄ alkenylene group, a C₂₋₄ alkynylene group or a C₁₋₄ heteroalkylene group, all of which groups may be substituted by one, two or more hydroxy or amino groups.

Preferred are compounds of formula (I), wherein A is a group of formula —CH₂—, —CH₂CH₂—, —OCH₂—, —OCH₂CH₂—, —SCH₂—, —SCH₂CH₂—, —S(═O)CH₂—, —SO₂CH₂—, —CH═CH—, —C≡C—, —CH(OH)CH(OH)— or —CH(NH₂)CH(OH)—.

Especially preferred are compounds of formula (I), wherein A is a single bond and Q is N.

Especially preferred are compounds of formula (I), wherein B and C are independently from each other —CH₂—, —CH₂CH₂— or —CH₂CH₂CH₂—.

Especially preferred are compounds of formula (I) wherein the residues are defined as above; A is —OCH₂— or —O—, Q is CR⁴, wherein R⁴ is —OR⁶, X is N, CH, CMe or C—OMe, Y is CH or CF, and R³ is cyclopropyl or X is CR⁵ and R⁵ and R² together form a bridge of the formula —O—CH₂—CH(Me)- or —O—CH₂CH₂—, B is —(CH₂)ₙ— and C is —(CH₂)ₘ— wherein n is 1, 2 or 3, and m is 1, 2 or 3, or wherein B and C are forming a heterobicyclic ring system, wherein Q is CR⁴ wherein R⁴ is H, or OR⁶, wherein R⁶ consists of glucose, mannose, allose, galactose, fructose, ribose, arabinose, xylose, streptose, apiose, trehalose, maltose, saccharose, lactose, dextrine, cyclodextrine, glycogen, starch, cellulose or a modified polysaccharides such as hydroxy ethyl starch (HES) or pegylated oligo- or polysaccharides or OR⁶ is a glucuronic acid, gluconic acid, or peracetylated glucuronic acid ester.

Especially preferred is a compound of formula (I) wherein R¹ is F, R² is a cyclopropyl group, X is CMe or N, Y is CH or CF, and n and m are independently from each other 1 or 2, R³ is CH₃C(═O)NH—, and R⁶ is a O-monosaccharide or OR⁶ is a glucuronic acid or a gluconic acid ester.

The present invention also relates to pharmacologically acceptable salts, or solvates and hydrates, respectively, and to compositions and formulations of compounds of formula (I). The present invention describes procedures to produce pharmaceutically useful agents, which contain these compounds, as well as the use of these compounds for the production of pharmaceutically useful agents.

The pharmaceutical compositions according to the present invention contain at least one compound of formula (I) as the active agent and optionally carriers and/or diluents and/or adjuvants. Optionally the pharmaceutical compositions according to the present invention may also contain additional known antibiotics.

Examples of pharmacologically acceptable salts of sufficiently basic compounds of formula (I) are salts of physiologically acceptable mineral acids like hydrochloric, hydrobromic, sulfuric and phosphoric acid; or salts of organic acids like methanesulfonic, p-toluenesulfonic, lactic, acetic, trifluoroacetic, citric, succinic, fumaric, maleic and salicylic acid. Further, a sufficiently acidic compound of formula (I) may form alkali or earth alkaline metal salts, for example sodium, potassium, lithium, calcium or magnesium salts; ammonium salts; or organic base salts, for example methylamine, dimethylamine, trimethylamine, triethylamine, ethylenediamine, ethanolamine, choline hydroxide, meglumin, piperidine, morpholine, tris-(2-hydroxyethyl)amine, lysine or arginine salts. Compounds of formula (I) may be solvated, especially hydrated. The hydratization can occur during the process of production or as a consequence of the hygroscopic nature of the initially water free compounds of formula (I). The compounds of formula (I) contain asymmetric C-atoms and may be present either as achiral compounds, mixtures of diastereomers, mixtures of enantiomers or as optically pure compounds.

The present invention also relates to prodrugs which are composed of a compound of formula (I) and at least one pharmacologically acceptable protective group which are bound to a normally free carboxy or hydroxy group of a mono-, di-, oligo- or polysaccharide and which will be cleaved off under physiological conditions, such as an alkoxy-, aralkyloxy-, acyl-, —SO₃H, —PO₃H₂, acyloxymethyl group (e.g. pivaloyl-oxymethyl), a 2-alkyl-, 2-aryl- or 2-aralkyl-oxycarbonyl-2-alkylidene ethyl group or a acyloxy group as defined herein, e.g. ethoxy, benzyloxy, acetyl or acetyloxy.

As mentioned above, therapeutically useful agents that contain compounds of formula (I), their solvates, salts or formulations are also comprised in the scope of the present invention. In general, compounds of formula (I) will be administered by using the known and acceptable modes known in the art, either alone or in combination with any other therapeutic agent. Such therapeutically useful agents can be administered by one of the following routes: oral, e.g. as tablets, dragees, coated tablets, pills, semisolids, soft or hard capsules, for example soft and hard gelatine capsules, aqueous or oily solutions, emulsions, suspensions or syrups, parenteral including intravenous, intramuscular and subcutaneous injection, e.g. as an injectable solution or suspension, rectal as suppositories, by inhalation or insufflation, e.g. as a powder formulation, as microcrystals or as a spray (e.g. liquid aerosol), transdermal, for example via an transdermal delivery system (TDS) such as a plaster containing the active ingredient or intranasal. For the production of such tablets, pills, semisolids, coated tablets, dragees and hard, e.g. gelatine, capsules the therapeutically useful product may be mixed with pharmaceutically inert, inorganic or organic excipients as are e.g. lactose, sucrose, glucose, gelatine, malt, silica gel, starch or derivatives thereof, talc, stearinic acid or their salts, dried skim milk, and the like. For the production of soft capsules one may use excipients as are e.g. vegetable, petroleum, animal or synthetic oils, wax, fat, polyols. For the production of liquid solutions, emulsions or suspensions or syrups one may use as excipients e.g. water, alcohols, aqueous saline, aqueous dextrose, polyols, glycerine, lipids, phospholipids, cyclodextrins, vegetable, petroleum, animal or synthetic oils. Especially preferred are lipids and more preferred are phospholipids (preferred of natural origin; especially preferred with a particle size between 300 to 350 nm) preferred in phosphate buffered saline (pH=7 to 8, preferred 7.4). For suppositories one may use excipients, as are e.g. vegetable, petroleum, animal or synthetic oils, wax, fat and polyols. For aerosol formulations one may use compressed gases suitable for this purpose, as are e.g. oxygen, nitrogen and carbon dioxide. The pharmaceutically useful agents may also contain additives for conservation, stabilisation, e.g. UV stabilizers, emulsifiers, sweetener, aromatisers, salts to change the osmotic pressure, buffers, coating additives and antioxidants.

A daily dosage per patient of about 1 mg to about 4000 mg especially about 50 mg to 3 g is usual with those of ordinary skill in the art appreciating that the dosage will depend also upon the age, conditions of the mammals, and the kind of diseases being treated or prevented. The daily dosage can be administrated in a single dose or can be divided over several doses. An average single dose of about 50 mg, 100 mg, 250 mg, 500 mg, 1000 mg and 2000 mg can be contemplated.

The compounds of the formula (I) can be synthesized according to a general reaction scheme starting from commercially available and custom starting materials, whereby a 8-chloro-4-oxo-4H-quinolizine-3-carboxylic acid derivative or a 2-chloro-6-oxo-6H-pyrido[1,2-a]pyrimidine-7-carboxylic acid ester derivative of formula (II) is coupled to an oxazolidinone derivative of formula (III) that comprises a nucleophilic NH group:

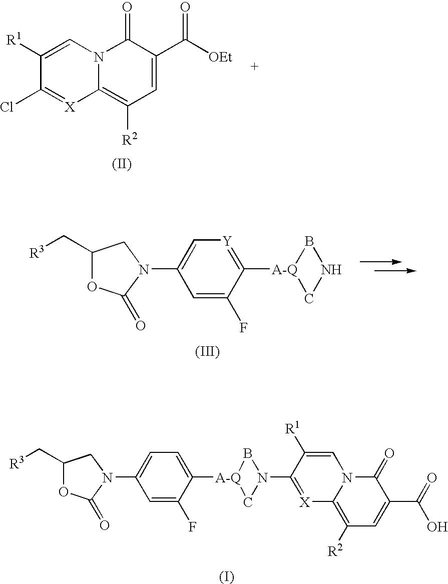

This reaction is carried out by refluxing an excess of the amine component (III) and the corresponding pyridin-2-one (II) in an anhydrous solvent such as acetonitrile in the presence of a base such as sodium bicarbonate. Alternatively a solvent such as N-methylpyrrolidinone and trimethylsilyl chloride as a catalyst and potassium carbonate or Hünigs base at elevated temperature is used. The free acid of formula (I) is than obtained from the mixture of the above product by treatment with lithium hydroxide in a tetrahydrofurane/water mixture at elevated temperature. Alternatively, instead of the ethyl ester of compound (II) the corresponding benzyl ester can be used, which is than removed by hydrogenolysis.
The synthesis of the required 2-chloro-6-oxo-6H-pyrido[1,2-a]pyrimidine-7-carboxylic acid as well as 8-chloro-4-oxo-4H-quinolizine-3-carboxylic acid derivatives of formula (II) has been described extensively in J. Med. Chem. 1996, 39, 3070-3088; Bioorg. Med. Chem. Lett. 1997, 7, 1167-1170; U.S. Pat. No. 5,789,591 (1998); U.S. Pat. No. 5,693,813 (1997); Heterocycles 1999, 51, 1345-1353.
Oxazolidinone derivatives of formula (III) may be prepared according to the following scheme that uses commercially available 1-nitro-3,4-difluoro arenes that are reacted with a corresponding nucleophilic linker moiety using Hünigs base in dimethylformamide or acetonitrile at elevated temperature:

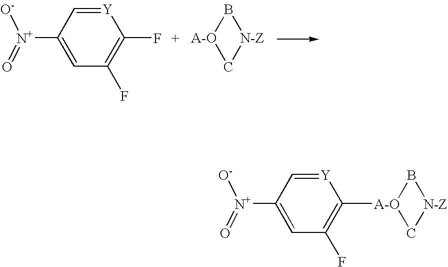

The resulting product is than reduced using iron/ammonium chloride under reflux in a water/ethanol mixture. The required oxazolidinone derivatives of formula (III) are than formed by reacting the free amino group with lithium tert-butoxide in methanol/DMF and 2-substituted acetic acid 1-chloromethylethyl ester, for example (S)-acetic acid 2-acetylamino-1-chloromethylethyl ester. Finally, the protecting group Z is removed, for example by treatment with trifluoroacetic acid in dichloromethane in case of a Boc protecting group.
Oxazolidinone derivatives of formula (III) may be also prepared according to the following scheme that uses aryl phenols as starting materials. Such aryl phenol is reacted in step i.) under water free conditions with a protected electrophilic building block that is substituted with a leaving group such as mesylate or halogen, in a solvent such as acetone or dimethylformamide, sodium hydride or potassium carbonate as a base to aid the nucleophilic substitution. Step ii.) The protecting group Z (for example the carboxybenzyloxy group) is removed in step ii.), for example by treatment with hydrogen and palladium in a solvent such as methanol, ethanol and tetrahydrofurane:

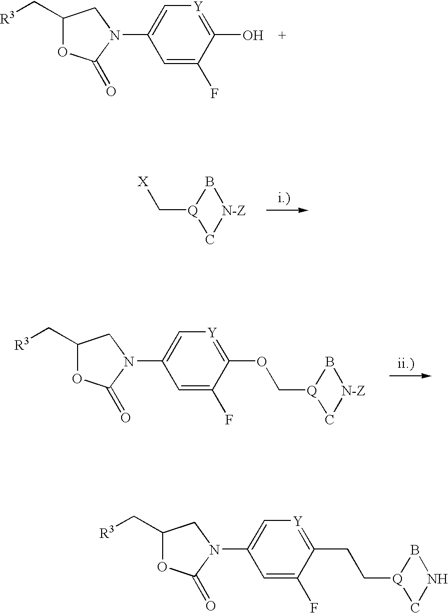

In case the final compound of formula (I) contains a saccharide, either Q, B or C may contain a protected hydroxy group. After coupling the respective building blocks of formula (II) and (III) compounds the protecting group W is removed during step iii.) according to the following example, if this has not happened under step ii.), for example by using hydrogen and palladium on charcoal if W is benzyl. In the step iv.), an activated natural or unnatural substituted or unsubstituted mono-, di-, oligo- or polysaccharide is than coupled to the free hydroxy group of the resulting hybrid molecule, for example by using glucose-1-O-trichloroimidate in dichloro methane and BF₃-Et₂O and/or TMSOTf as catalyst, resulting in an overall yield of 70-90% the desired final product.
The following scheme shows a corresponding example, in which the protected hydroxy group is contained in Q:

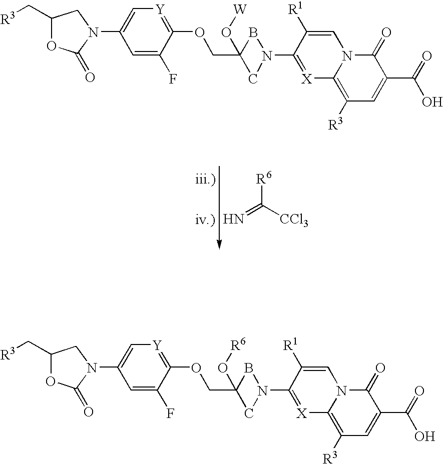

In the coupling step iv.), protected mono-, di-, oligo-, and polysaccharides as well as peracetylated or perbenzylated saccharides may be used to form the final ether bond. These protected sugar residues may be de-protected in step v.) by hydrogenolysis in case of benzylic groups or by basic cleavage in case of acetyl groups. Alternatively, step iv.) may be carried out by using an activated saccharide acid as for example 2,2,7,7-tetramethyl-tetrahydro-bis[1,3]dioxolo[4,5-b;4′,5′-d]pyran-5-carboxylic acid 4-nitrophenolic ester to form compounds of formula (I) where OR⁶ is forming an saccharide ester bond. In these cases, as a last deprotecting step v.) the acetone protecting groups are removed under acidic conditions.

### Example 1

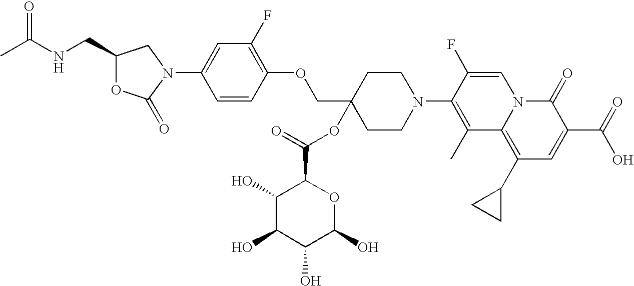

8-[4-{4-[(S)-5-(Acetylamino-methyl)-2-oxo-oxazolidin-3-yl]-2-fluoro-phenoxymethyl}-4-((2S,3S,4S,5R,6R)-3,4,5,6-tetrahydroxy-tetrahydro-pyran-2-carbonyloxy)-piperidin-1-yl]-1-cyclopropyl-7-fluoro-9-methyl-4-oxo-4H-quinolizine-3-carboxylic acid. C38H42F2N4O14 (816.77), MS 817.5 (M+H)⁺ Method ESI⁺.

### Example 2

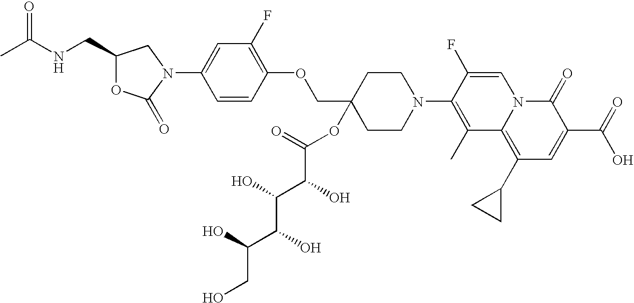

8-[4-{4-[(S)-5-(Acetylamino-methyl)-2-oxo-oxazolidin-3-yl]-2-fluoro-phenoxymethyl}-4-((2R,3S,4R,5R)-2,3,4,5,6-pentahydroxy-hexanoyloxy)-piperidin-1-yl]-1-cyclopropyl-7-fluoro-9-methyl-4-oxo-4H-quinolizine-3-carboxylic acid. C38H44F2N4O14 (818.80), MS 819.6 (M+H)⁺ Method ESI⁺.

### Example 3

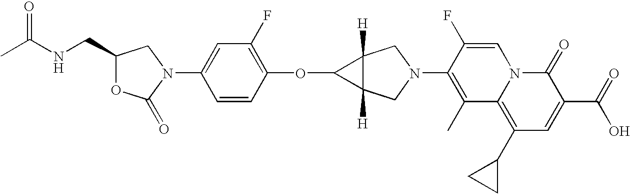

8-((1R,5S)-6-{4-[(S)-5-(Acetylamino-methyl)-2-oxo-oxazolidin-3-yl]-2-fluoro-phenoxy}-3-aza-bicyclo[3.1.0]hex-3-yl)-1-cyclopropyl-7-fluoro-9-methyl-4-oxo-4H-quinolizine-3-carboxylic acid. C31H30F2N4O7 (608.60), MS 609.4 (M+H)⁺ Method ESI⁺.

### Example 4

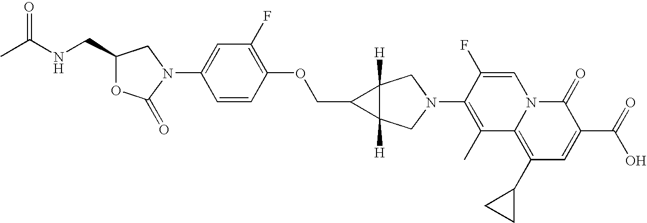

8-((1S,5R)-6-{4-[(S)-5-(Acetylamino-methyl)-2-oxo-oxazolidin-3-yl]-2-fluoro-phenoxymethyl}-3-aza-bicyclo[3.1.0]hex-3-yl)-1-cyclopropyl-7-fluoro-9-methyl-4-oxo-4H-quinolizine-3-carboxylic acid. C32H32F2N4O7 (622.63), MS 623.4 (M+H)⁺ Method ESI⁺.

### Example 5

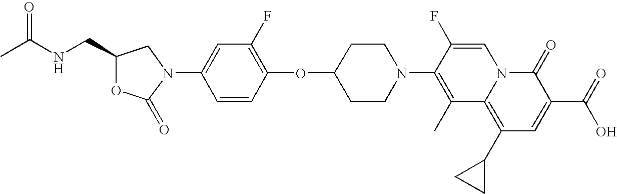

8-(4-{4-[(S)-5-(Acetylamino-methyl)-2-oxo-oxazolidin-3-yl]-2-fluoro-phenoxy}-piperidin-1-yl)-1-cyclopropyl-7-fluoro-9-methyl-4-oxo-4H-quinolizine-3-carboxylic acid. C31H32F2N4O7 (610.62), MS 611.3 (M+H)⁺ Method ESI⁺.

### Example 6

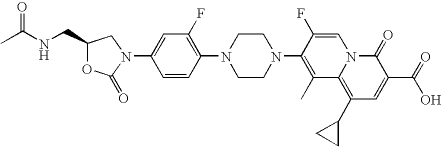

8-(4-{4-[(S)-5-(Acetylamino-methyl)-2-oxo-oxazolidin-3-yl]-2-fluoro-phenyl}-piperazin-1-yl)-1-cyclopropyl-7-fluoro-9-methyl-4-oxo-4H-quinolizine-3-carboxylic acid. C30H31F2N5O6 (595.61), MS 596.4 (M+H)⁺ Method ESI⁺.

### Example 7

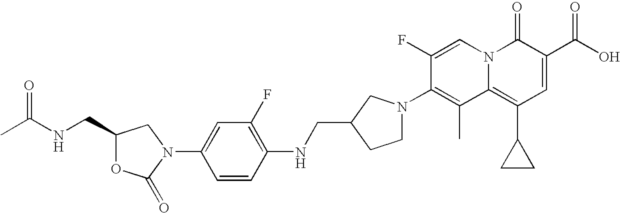

8-[3-({4-[(S)-5-(Acetylamino-methyl)-2-oxo-oxazolidin-3-yl]-2-fluoro-phenylamino}-methyl)-pyrrolidin-1-yl]-1-cyclopropyl-7-fluoro-9-methyl-4-oxo-4H-quinolizine-3-carboxylic acid. C31H33F2N5O6 (609.64), MS 610.5 (M+H)⁺ Method ESI⁺.

### Example 8

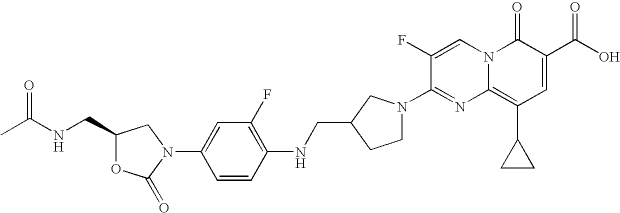

2-[3-({4-[(S)-5-(Acetylamino-methyl)-2-oxo-oxazolidin-3-yl]-2-fluoro-phenylamino}-methyl)-pyrrolidin-1-yl]-9-cyclopropyl-3-fluoro-6-oxo-6H-pyrido[1,2-a]pyrimidine-7-carboxylic acid. C29H30F2N6O6 (596.60), MS 597.3 (M+H)⁺ Method ESI⁺.

### Example 9

All examples were tested against a panel of Gram positive and Gram negative bacteria. With the exception of the saccharide modified compounds (such as example 1 and 2) which are inactive in the in vitro screens, the compounds exhibit a broader and more pronounced activity than the corresponding quinolone and oxazolidinone as well as the 1:1 combination of these compounds.
Typical MIC ranges (mg/ml) are:
S. aureus (MRSA) 0.06-2 (linezolid 1-2, ciprofloxacin 0.5-32)
S. aureus (MSSA) 0.06-2 (linezolid 1-2, ciprofloxacin 0.125-1)
E. faecalis 0.02-1 (linezolid 0.5-2, ciprofloxacin 0.5-32)
E. faecium 0.02-1 (linezolid 1-2, ciprofloxacin 0.25-32)
S. pneumoniae 0.02-1 (linezolid 0.125-1, ciprofloxacin 1-4)
However, cleavage of saccharide modified compounds such as compounds from example 1 and 2 by esterases and glucosidases that occur in human plasma yields the corresponding antibiotically active compounds.

### REFERENCES

The references mentioned herein are all expressly incorporated by reference.

While certain embodiments of the present invention have been described and/or exemplified above, various other embodiments will be apparent to those skilled in the art from the foregoing disclosure. The present invention is, therefore, no limited to the particular embodiments described and/or exemplified, but is capable of considerable variation and modification without departure from the scope of the appended claims.

1. A compound having the structural formula (I) 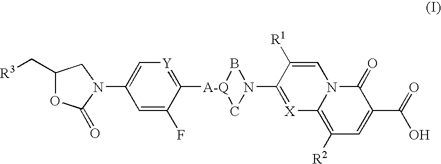
and its stereoisomers or its pharmacologically acceptable salt, solvate, hydrate, or formulation thereof,
wherein
R¹ is H or F; and
R² is an alkyl group, an alkenyl group, an alkynyl group, a heteroalkyl group, a cycloalkyl group, a heterocyclo-alkyl group, an aryl group, a heteroaryl group, an alkyl-aryl group or a heteroarylalkyl group; all of which may be substituted with one, two or more halogen atoms like F or Cl or hydroxy or amino groups; and
R³ is an azido, or a C₁₋₆-heteroalkyl group, a heteroarylalkyl group, a heteroarylcycloalkyl group or a heteroalkylheteroaryl group; and
A is a single bond, O, S, S(═O), SO₂ or an alkylene group, an alkenylene group, an alkynylene group, a heteroalkylene group, a cycloalkylene group, a heterocycloalkylene group, an arylene group or a heteroarylene group all of which groups may be substituted; and
B and C are independent from each other alkylene, alkenylene, alkynylene or heteroalkylene, whereby Q-B—N—C are forming together a heterocycloalkyl group or a bicyclic heterocycloalkyl group, all of these groups may be substituted with one or more R⁴ groups; and
Q is CR⁴ or N; and
X is CR⁵ or N; and
Y is CH, CF or N; and
R⁴ is H, OR⁶, a group of formula —OPO₃R⁶₂ or —OSO₃R⁶ or an alkyl group or a heteroalkyl group carrying one or more OR⁶, —OPO₃R⁶₂ or —OSO₃R⁶ group(s), wherein the groups R⁶ independently of each other are H, an ether or an ester of a natural or unnatural, substituted or unsubstituted monosaccharide, a natural or unnatural, substituted or unsubstituted disaccharide, a natural or unnatural, substituted or unsubstituted oligosaccharide or a natural or unnatural, substituted or unsubstituted polysaccharide.
R⁵ is H, CH₃, OCH₃, F, Cl, OH, NH₂, —CN, an alkyl group or a heteroalkyl group, and
R² and R⁵ can be linked via an alkylene, an alkenylene or a heteroalkylene group or be a part of a cycloalkylene or heterocycloalkylene group, in case R² is not H and R⁵ is not H, CH₃, OCH₃, F, Cl, OH, NH₂, —CN; or a pharmacologically acceptable salt, solvate, hydrate or formulation thereof.

2. The compound according to claim 1 wherein R² is selected from a methyl group, an ethyl group, a 2-propyl group, a C₃-C₆-cycloalkyl, a phenyl or a pyridyl group; all of which may be substituted with one, two, three or more fluorine atoms.

3. The compound according to claim 1 wherein R1 is F, R2 is a cyclopropyl group and X is N or CMe.

4. The compound according to claim 1, wherein R⁴ is selected independently from H, OR⁶ or a heteroalkyl group which contains one or more OR⁶ groups wherein the groups R⁶ are selected independently from each other glucose, glucosamine, mannose, allose, galactose, fructose, ribose, arabinose, xylose, streptose, apiose, trhalose, maltose, saccharose, lactose, dextrine, cyclodextrine, glycogen, starch, cellulose or a modified polysaccharide such as e.g. hydroxyethyl starch or a pegylated oligo- or polysaccharide.

5. The compound according to claim 1 wherein R⁴ is OR⁶ and OR⁶ is an ester of selected from a group of glucuronic acid, mannopyranuronic acid, gluco-pyranosiduronic acid, tartaric acid, xylaric acid, or galactaric acid.

6. The compound according to claim 1 wherein R³ is selected independently from a group of —NHCOCH═CHAryl, heteroaryl such as unsubstituted 1,2,3-triazol or 1,2,3-triazol substituted by F, Cl or Me, oxa-3-oxazole, —NHSO₂Me, —NHSOMe, —NHCOOMe, —NHCOMe, —NHCS₂Me, —NHCSMe, —NHCSNH₂, or —NHCSOMe.

7. The compound according to claim 1, wherein R³ is independently selected from a group of —NHCOMe, —NHCSMe, —NHCOCHF₂, or —NHCOCHCl₂.

8. The compound according to claim 1, wherein Y is independently selected from N, CH, CF, CCl or the CMe group, which may be substituted by one, two or three fluorine atoms.

9. The compound according to claim 1, wherein A is independently selected from O or a group selected from —CH₂—, —CH₂CH₂—, —OCH₂—, —OCH₂CH₂—, —SCH₂—, —SCH₂CH₂—, —S(═O)CH₂—, —SO₂CH₂—, —CH═CH—, —C≡C—, —CH(OH)CH(OH)— or —CH(NH₂)CH(OH)—.

10. The compound according to claim 1, wherein B and C are independently from each other selected from —CH₂—, —CH₂CH₂—, —CH(−)CH₂— whereby a heterobicyclic system is formed, and Q is independently selected from CH, N or COR⁶.

11. Pharmaceutical compositions containing a compound according to claim 1 and optional carriers and/or adjuvants and/or diluents for the preparation of medicaments for the treatment of bacterial infections.

12. Pro-drugs, which contain a compound according to claim 1 and at least one pharmacologically acceptable protective group for the preparation of medicaments for the treatment of bacterial infections.

13. A method for the treatment of bacterial infections in a patient in need thereof, said method comprising administering to said patient a therapeutically effective amount of a compound according to claim 1.
